(19) United States
(12) Patent Application Publication
(10) Pub. No.: US 2024/0299702 A1
(43) Pub. Date: Sep. 12, 2024

(54) ELONGATED MEDICAL BODY AND METHOD FOR MANUFACTURING ELONGATED MEDICAL BODY

(71) Applicant: TERUMO KABUSHIKI KAISHA, Shibuya-ku Tokyo (JP)

(72) Inventors: Nozomu Watanabe, Kanagawa (JP); Yuya OTAKE, Shizuoka (JP)

(73) Assignee: TERUMO KABUSHIKI KAISHA, Shibuya-ku Tokyo (JP)

(21) Appl. No.: 18/593,148

(22) Filed: Mar. 1, 2024

(30) Foreign Application Priority Data
Mar. 6, 2023 (JP) 2023-033578

(51) Int. Cl. A61M 25/00; A61M 25/06

(52) U.S. Cl. CPC A61M 25/005; A61M 25/0009; A61M 2025/0047; A61M 2025/0681; A61M 2205/587

(57) ABSTRACT

An elongated medical body having a resistance that is constantly 10 gf or less when a tube is moved 100 times while the elongated medical body is clamped at 500 gf between a pair of abutting members.

### CROSS-REFERENCES TO RELATED APPLICATIONS

This application is based on and claims priority to Japanese Patent Application No. 2023-033578 filed on Mar. 6, 2023, the entire content of which is incorporated herein by reference.

### TECHNOLOGICAL FIELD

The present invention generally relates to an elongated medical body and a method for manufacturing an elongated medical body.

### BACKGROUND DISCUSSION

A guiding catheter (also referred to as an elongated medical body) is a catheter for guiding a balloon catheter or the like that is inserted into a target site to treat the heart or a lower limb artery. An example is disclosed in International Patent Application Publication No. 2018/092387 (WO 2018/092387 A).

Particularly, a guiding catheter used for bringing a balloon catheter or the like from the radial artery at the wrist to the iliac artery (or a lower limb artery much closer to a distal part) requires favorable pushability and trackability and also a small resistance due to friction, that is, what is called surface lubricity, since the guiding catheter has a large outside diameter and passes through linear regions, bent regions, and calcified regions.

### SUMMARY

An effective solution for providing favorable pushability, trackability, and surface lubricity to a guiding catheter is to reduce the resistance due to friction between the guiding catheter and the wall of a blood vessel. However, when a long guiding catheter with a large outside diameter is navigated from the radial artery at the wrist to a lower limb artery, the guiding catheter travels a long distance inside a blood vessel and has a large external surface area, whereby a surface area of a hydrophilic coating for reducing a frictional force between the guiding catheter and the wall of the blood vessel also becomes large. Such an increase in surface area of the hydrophilic coating may decrease the surface lubricity due to friction at meandering portions or calcified sites and may increase the number of fine particles generated from the hydrophilic coating.

Disclosed here is an elongated medical body which has favorable pushability and trackability and maintains surface lubricity, that is, a small resistance due to friction, or does not decrease in surface lubricity even when subjected to friction and also reduces the number of fine particles generated.

The elongated medical body has such characteristics and attributes is achieved by the following techniques.
(1) An elongated medical body having a resistance that is constantly 10 gf or less when the elongated medical body is moved 100 times while the elongated medical body is clamped at 500 gf between a pair of abutting members.
(2) The elongated medical body according to (1), wherein the elongated medical body is a tube.
(3) The elongated medical body according to (2), wherein the tube includes: a lubricant layer containing a lubricating resin to which a first non-lubricating resin is added; and an outer layer coated with the lubricant layer and containing a second non-lubricating resin, and a ratio of the first non-lubricating resin added to the lubricating resin ranges from 1/1000 to 1.
(4) The elongated medical body according to any one of (1) to (3), wherein the elongated medical body is a guiding catheter for lower limb artery.
(5) The elongated medical body according to (3) or (4), wherein the first non-lubricating resin is insoluble in water.
(6) The elongated medical body according to any one of (3) to (5), wherein the first non-lubricating resin is a vinyl chloride resin or a urethane resin.
(7) The elongated medical body according to any one of (3) to (6), wherein the second non-lubricating resin is a polyamide elastomer or a polyamide.
(8) The elongated medical body according to (1), wherein the elongated medical body includes: a lubricant layer containing a lubricating resin to which a first non-lubricating resin is added; and a metallic member inside the lubricant layer, and a ratio of the first non-lubricating resin added to the lubricating resin ranges from 1/1000 to 1.
(9) The elongated medical body according to (8), wherein the metallic member is SUS or Ni—Ti.
(10) The elongated medical body according to (9), wherein the elongated medical body is a guidewire.
(11) The elongated medical body according to any one of (3) to (10), wherein the lubricating resin is hydrophilic.
(12) The elongated medical body according to any one of (3) to (11), wherein the lubricating resin is an acrylic resin.
(13) A method for manufacturing an elongated medical body, the method including:
coating an outer layer containing a second non-lubricating resin with a solution containing a first non-lubricating resin and a lubricating resin in which a mass ratio of the first non-lubricating resin added to the lubricating resin ranges from 1/1000 to 1; and heating at 110° C. or less for 6 hours or less to form a lubricant layer on periphery of the outer layer.
(14) The method for manufacturing an elongated medical body according to (13), wherein the lubricant layer is formed on periphery of the outer layer by heating for 1 hour or less.

The elongated medical body with the above configuration has favorable pushability and trackability since a resistance is as low as 10 gf or less when the tube is moved while clamped at 500 gf with a testing machine (to be described). In addition, since the resistance is maintained at 10 gf or less even after the tube is moved 100 times while clamped at 500 gf, the elongated medical body has high durability and reduces the number of fine particles generated. Accordingly, there is provided an elongated medical body which has favorable pushability and trackability and maintains surface lubricity, that is, a small resistance due to friction, or does not decrease in surface lubricity even when subjected to friction and also reduces the number of fine particles generated.

According to another aspect, an elongated medical body possesses a radially inwardly facing inner peripheral surface and a radially outwardly facing outer peripheral surface, wherein the elongated medical body comprises: an elongated tubular body possessing a proximal end and a distal end, with the elongated tubular body having a lumen extending throughout a length of the elongated tubular body and opening to both an open distal end of the elongated tubular body at the distal end of the elongated tubular body and an open proximal end of the elongated tubular body at the proximal end of the elongated tubular body. The elongated tubular body includes a first layer extending along the length of the elongated tubular body. The elongated tubular body also includes a second layer located at least at a distal end portion of the elongated tubular body, inclusive of the distal end of the elongated tubular body. The second layer is positioned radially outwardly of the first layer so that the second layer is positioned in overlying relation to and covers the first layer. The second layer has an outer surface forming at least a part of the outer peripheral surface of the elongated medical body. The second layer is a lubricant layer comprised of a non-lubricating resin and a lubricating resin.

### BRIEF DESCRIPTION OF THE DRAWINGS

FIG. 1 is an overall view of an elongated medical body according to an embodiment disclosed here.

FIG. 2 is a sectional front view of a tube in the elongated medical body according to this embodiment.

FIG. 3 is a schematic view of a testing device for conducting a wear test on the elongated medical body according to this embodiment, illustrating a state where the elongated medical body is clamped by a pair of abutting members.

FIG. 4 is a schematic view of the testing device for conducting a wear test on the elongated medical body according to this embodiment, illustrating a state where the elongated medical body is freed from the pair of abutting members.

FIG. 5 is a view illustrating resistances of medical elongated bodies according to Example and Comparative Example.

### DETAILED DESCRIPTION

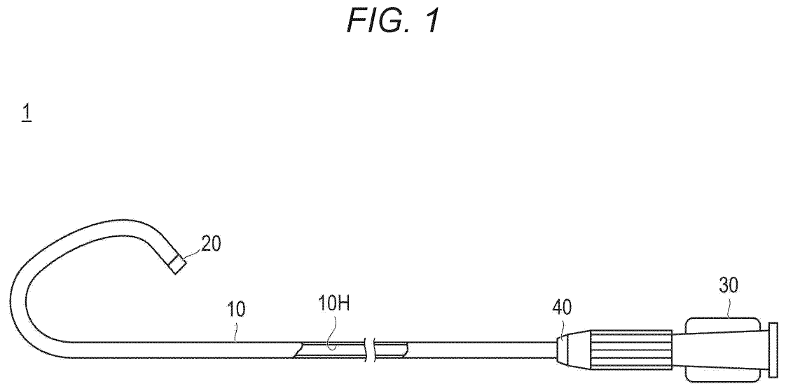

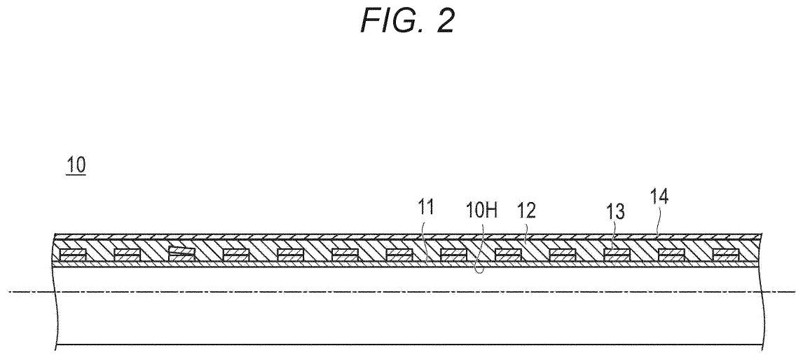

Hereinafter, an elongated medical body 1 and manufacturing method according to an embodiment, representing an example of the new elongated medical body and method for manufacturing an elongated medical body disclosed here, will be described with reference to FIGS. 1 and 2. FIG. 1 is an overall view of the elongated medical body 1 according to the embodiment disclosed by way of example. FIG. 2 is a sectional front view of a tube 10 in the elongated medical body 1 according to this embodiment.

The elongated medical body 1 according to this embodiment is a guiding catheter for lower limb artery used in surgery for diagnosing and treating a lesion in a blood vessel of the lower limb.

As illustrated in FIG. 1, the elongated medical body 1 includes a tube 10, a flexible distal tip 20 disposed on the distal side of the tube 10, and a hub (hub tube) 30 disposed on the proximal side of the tube 10. The distal side of the hub 30 is provided with a strain relief.

The tube 10 includes a tubular body having flexibility. A lumen 10H is formed in the tube 10 over the entire length of the tube 10. The lumen 10H is open at a distal end of the distal tip 20.

As illustrated in FIG. 2, the tube 10 includes an inner layer 11 disposed on the inner surface of the tube 10, an outer layer 12 disposed on the outer periphery of the inner layer 11, a lubricant layer 14 disposed at least on the distal side of the periphery of the outer layer 12, and a reinforcing material layer 13 disposed inside the outer layer 12.

The lubricant layer 14 includes a non-lubricating resin (first non-lubricating resin) and a lubricating resin. The outer layer 12 includes a second non-lubricating resin.

The lubricating resin in the lubricant layer 14 preferably is a hydrophilic lubricating resin. That is, the lubricating resin in the lubricant layer 14 contains a hydrophilic lubricating resin. For this reason, the lubricating resin is preferably a hydrophilic material. Examples of the hydrophilic material include block copolymers of glycidyl methacrylate-dimethylacrylamide.

In particular, the lubricating resin of the lubricant layer 14 is preferably an acrylic resin. The acrylic resin preferably contains a constitutional unit derived from a reactive monomer. Introduction of such a constitutional unit derived from a reactive monomer causes crosslinking or polymerization of polymers via an epoxy group, or the reactive monomer, thereby forming a network structure. Accordingly, the elongated medical body 1 maintains surface lubricity, that is, a small resistance due to friction, or does not decrease in surface lubricity even when subjected to friction and also reduces the number of fine particles generated.

The reactive monomer is preferably a (meth)acrylic acid ester having an epoxy group. In particular, the reactive monomer preferably contains at least one selected from the group consisting of glycidyl acrylate, glycidyl methacrylate (GMA), 3,4-epoxycyclohexyl methyl acrylate, 3,4-epoxycyclohexyl methyl methacrylate, and β-methyl glycidyl methacrylate because it is easy to control crosslinking or polymerization of polymers. A more preferable example of the reactive monomer is glycidyl (meth)acrylate, and a still more preferable example is glycidyl methacrylate (GMA). Herein, “(meth)acrylate” represents both acrylate and methacrylate. One type of the reactive monomer may be used independently or two or more types may be used in combination.

In addition, the acrylic resin preferably includes a constitutional unit (A) derived from a reactive monomer having an epoxy group and a constitutional unit (B) derived from a hydrophilic monomer. More preferably, the acrylic resin consists of the constitutional unit (A) derived from a reactive monomer having an epoxy group and the constitutional unit (B) derived from a hydrophilic monomer.

Examples of the hydrophilic monomer include acrylamide and derivatives thereof, vinylpyrrolidone, acrylic acid and methacrylic acid and derivatives thereof, polyethylene glycol acrylate and derivatives thereof, monomers having a sugar or a phospholipid in a side chain, and water-soluble monomers such as maleic anhydride, but the hydrophilic monomer is preferably N,N-dimethylacrylamide (DMAA). One type of the hydrophilic monomer may be used independently or two or more types may be used in combination.

A method for manufacturing the acrylic resin is not particularly limited. For example, a block copolymer having a hydrophilic moiety and a reactive moiety is produced by a method disclosed in JP 9-131396 A or the like.

As the first non-lubricating resin of the lubricant layer 14, a vinyl chloride resin (Polyvinyl chloride: PVC) or urethane resin is preferable.

Examples of the urethane resin include polyester-based urethane resins, polyether-based urethane resins, and polycarbonate-based urethane resins. A urethane elastomer is also employable, and a commercially available urethane elastomer having a polyether segment such as Pellethane 2363 available from Lubrizol or Elastollan available from BASF is preferable.

The first non-lubricating resin of the lubricant layer 14 is preferably insoluble in water. This configuration enhances the durability. The expression “insoluble in water” represents that a substance is insoluble (or hardly soluble) in water at room temperature (23° C.) and normal pressure (1 atmospheric pressure). For example, a substance is referred to as insoluble if less than 1 g of the substance dissolves in 100 ml of water at room temperature and normal pressure but not limited thereto.

A mass ratio of the first non-lubricating resin added to the lubricating resin preferably ranges from 1/1000 to 1.

Due to the addition of the first non-lubricating resin to the lubricating resin, when the tube 10 is moved 100 times while clamped at 500 gf (gram force) between a pair of abutting members 91 and 92 (to be described below), a resistance is constantly above 0 gf and 10 gf or less. Since the resistance when the tube 10 is moved while clamped at 500 gf is as low as 10 gf or less, the elongated medical body 1 has favorable pushability and trackability. In addition, since the resistance is maintained at 10 gf or less even after the tube 10 is moved 100 times while clamped at 500 gf, the elongated medical body 1 has high durability and maintains surface lubricity, that is, a small resistance due to friction, or does not decrease in surface lubricity even when subjected to friction and also reduces the number of fine particles generated.

Examples of a material for the second non-lubricating resin of the outer layer 12 include polyamide elastomers and/or polyamide resins, polyesters, polyester elastomers, polyurethane elastomers, and polyurethane resins having anti-kink properties and favorable pushability and trackability.

The reinforcing material layer 13 has a plurality of reinforcing wires for reinforcing the tube 10. Examples of the reinforcing wires include spiral or braided reinforcing wires. The reinforcing wires are made of metal such as stainless steel. As a specific example of the reinforcing wires, a stainless steel wire is pressed and processed into a flat plate to reduce the thickness of the tube 10 in the radial direction, and about 8 to 32 stainless steel wires are combined and wound or braided (braided member). The number of reinforcing wires is preferably a multiple of 8 for reinforcement in a tubular and balanced manner.

Such flat reinforcing wires receive an external stress uniformly as compared with ellipse reinforcing wires (i.e., reinforcing wires having an elliptical transverse cross-sectional shape), whereby the physical properties become stable.

The inner layer 11 preferably includes a material that reduces friction at least at a portion that comes into contact with a device, such as an operating catheter and a guidewire, when the device is inserted through the lumen 10H. This configuration enables the device inserted through the tube 10 to move in the longitudinal direction with a smaller sliding resistance, which enhances operability. A specific example of the material for the inner layer 11 includes a fluororesin material such as polytetrafluoroethylene (PTFE).

The number of layers included in the tube 10 and the material for each layer may be changed along the longitudinal direction of the tube 10. For example, in order to make a distal portion of the tube 10 more flexible, the number of layers may be reduced, or a more flexible material may be used, or a reinforcing material may not be disposed in the portion.

Since the elongated medical body 1 is inserted into a body while the position of the elongated medical body 1 is monitored by X-ray fluoroscopy, a radiopaque material (radiopaque medium) is preferably blended in the material for the outer layer 12. Examples of the radiopaque material include barium sulfate, bismuth oxide, and tungsten.

The radiopaque material is not necessarily present over the entire length of the tube 10 and may be present in a part of the tube 10, for example, in the distal portion or in the distal tip 20.

The distal portion of the tube 10 is curved in a desired shape suitable for a site such as the left coronary artery and the right coronary artery into which the distal portion of the tube 10 is inserted as shown in FIG. 1. In particular, the distal portion has a shape that facilitates an operation of engaging with the coronary ostium or a shape that more reliably maintains the state of engagement with the coronary ostium, but the distal portion may not have a particular shape as long as it is suitable for a lower limb artery.

Furthermore, the distal tip 20 is coupled to a distal end of the tube 10. The distal tip 20 includes a material rich in flexibility and has the distal end preferably having a rounded shape. With such a distal tip 20, it is possible to travel smoothly and safely inside a curved, bent, or branched blood vessel. Examples of the material for the distal tip 20 include various rubber materials such as natural rubber, isoprene rubber, butadiene rubber, chloroprene rubber, silicone rubber, fluororubber, and styrene-butadiene rubber and various thermoplastic elastomers such as styrene-based, polyolefin-based, polyurethane-based, polyester-based, polyamide-based, polybutadiene-based, trans-polyisoprene-based, fluorine-based, and chlorinated polyethylene-based elastomers, but the material for the distal tip 20 is preferably a polyamide elastomer.

In addition, the aforementioned radiopaque material (radiopaque medium) may be blended in the material for the distal tip 20.

The distal tip 20 is not particularly limited in length but preferably has a length of about 0.5 to 3 mm, and more preferably, about 1 to 2 mm.

The hub 30 is attached (fixed) to a proximal end of the tube 10. The hub 30 is provided with an inner cavity that communicates with the lumen 10H. This inner cavity has an inside diameter substantially equal to the inside diameter of the lumen 10H and is linked to or connected to (transitions to) the inner surface of a proximal portion of the lumen 10H without causing a step or the like.

From the hub 30, for example, an elongated object (linear body) such as guidewire, catheter of any kind (for example, balloon catheter and stent delivery catheter), endoscope, ultrasonic probe, and temperature sensor is inserted or removed, and a liquid of any kind such as contrast medium (radiopaque medium), medicinal solution, or physiological saline is injected. Furthermore, the hub 30 is connectable to other instruments such as Y-shaped branch connector.

Hereinafter described is a method for manufacturing the elongated medical body 1 according to this embodiment.

The first step is to prepare an elongated medical body including the outer layer 12 that contains the second non-lubricating resin.

The next step is to prepare a coating liquid containing the first non-lubricating resin and the lubricating resin.

A solvent for dissolving or dispersing the first non-lubricating resin and the lubricating resin is not particularly limited as long as it dissolves the first non-lubricating resin and the lubricating resin. Specific examples of the solvent include, but are not particularly limited to, water, alcohols such as methanol, ethanol, isopropanol, and ethylene glycol, ketones such as acetone, methyl ethyl ketone, and cyclohexanone, esters such as ethyl acetate, halides such as chloroform, olefins such as hexane, ethers such as tetrahydrofuran (THF) and butyl ether, aromatics such as benzene and toluene, amides such as N,N-dimethylformamide (DMF), and sulfoxides such as dimethyl sulfoxide (DMSO). These solvents may be used independently or two or more types may be used in combination. Among the examples, N,N-dimethylformamide (DMF) is preferable.

Next, the outer layer 12 containing the second non-lubricating resin is coated with or immersed in the solution obtained by adding the first non-lubricating resin to the lubricating resin.

The resultant is heated at 110° C. or less for 6 hours or less (preferably 1 hour or less), thereby forming the lubricant layer 14 on the periphery of the outer layer 12 containing the second non-lubricating resin. Through these steps, the elongated medical body 1 according to this embodiment is manufactured. The heating temperature is preferably 90 to 150° C., and more preferably, 100 to 140° C. In particular, a temperature of from 100 to 130° C. shortens the time of heat treatment and enables crosslinking without applying an excessive heat load to a base material layer, and it is possible to provide lubricity even to a base material with relatively low heat resistance or to control the lubricity more easily. The temperature may be changed during the heat treatment.

Furthermore, the time for drying or heat treatment is not particularly limited but is preferably 30 minutes to 30 hours, more preferably, 1 to 25 hours, and particularly preferably, 1 to 6 hours. Such a time range effectively promotes polymerization and forms a strong outer layer. Accordingly, it is possible to maintain high lubricity (surface lubricity) for a longer period of time. In addition, the above time range prevents excessive progress of crosslinking or polymerization. Therefore, it is possible to prevent reduction in swelling behavior attributed to excessive curing of the lubricant layer, thereby maintaining good lubricity (surface lubricity).

In this step, it is preferable to perform the heat treatment after drying treatment from the aspect of particularly effective (efficient) promotion of crosslinking or polymerization. That is, the method here preferably includes a drying treatment in advance of the heat treatment. Going through such drying treatment in addition to the heat treatment further improves the effect of promoting crosslinking or polymerization of polymers because the heat treatment is performed in a state where the solvent is distilled away. Since the heat treatment is performed in a shorter time, even a polymer material that is easily deformed or plasticized by heat is employable as the base material layer.

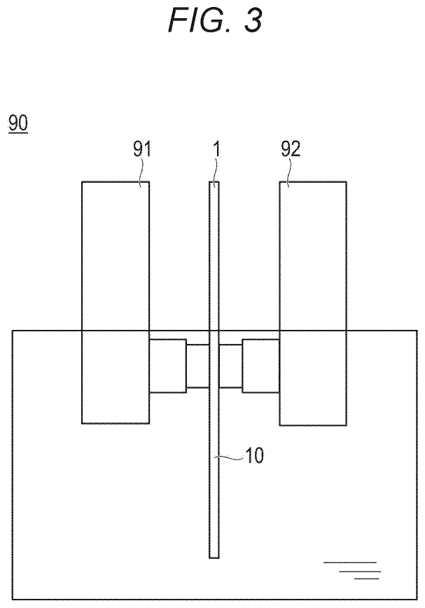

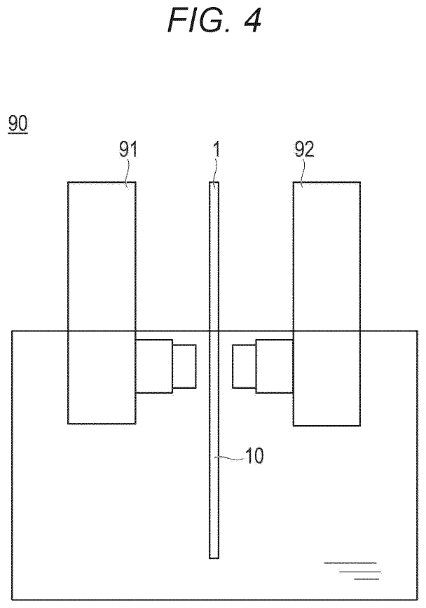

With reference to FIGS. 3 and 4, hereinafter described is a testing device 90 for conducting a wear test on the elongated medical body 1 according to this embodiment. FIG. 3 is a schematic view of the testing device 90 for conducting a wear test on the elongated medical body 1 according to this embodiment, illustrating a state where the elongated medical body 1 is clamped by the pair of abutting members 91 and 92. FIG. 4 is a schematic view of the testing device 90 for conducting a wear test on the elongated medical body 1 according to this embodiment, illustrating a state where the elongated medical body 1 is freed from the pair of abutting members 91 and 92.

As illustrated in FIGS. 3 and 4, the testing device 90 includes the pair of abutting members 91 and 92. The pair of abutting members 91 and 92 is configured to approach and separate from each other. When the pair of abutting members 91 and 92 approaches each other, the elongated medical body 1 into which a cored bar is inserted is clamped with a predetermined force by the abutting members 91 and 92. An example of the testing device 90 includes DL1000 available from OakRiver Technology.

Hereinafter described is a method for measuring a resistance with the testing device 90.

First, the elongated medical body 1 is set in the testing device 90 in water. The water is at room temperature.

Next, the pair of abutting members 91 and 92 is brought close to each other, and the elongated medical body 1 made of silicone with 60 Shore A is clamped at 500 gf with pads having a height of 12.35 mm and a width of 31.75 mm.

Next, the resistance is measured while the elongated medical body 1 is pulled up at a predetermined speed. The elongated medical body 1 is pulled up at a rate of, for example, 8.3 mm/s, and a pulling range is, for example, 25 mm.

Next, the pair of abutting members 91 and 92 is separated from each other to release the clamping state. The elongated medical body 1 is returned to the initial position.

The above steps are repeated a predetermined number of times, and the resistance is measured the predetermined number of times. The number of measurements is, for example, 100.

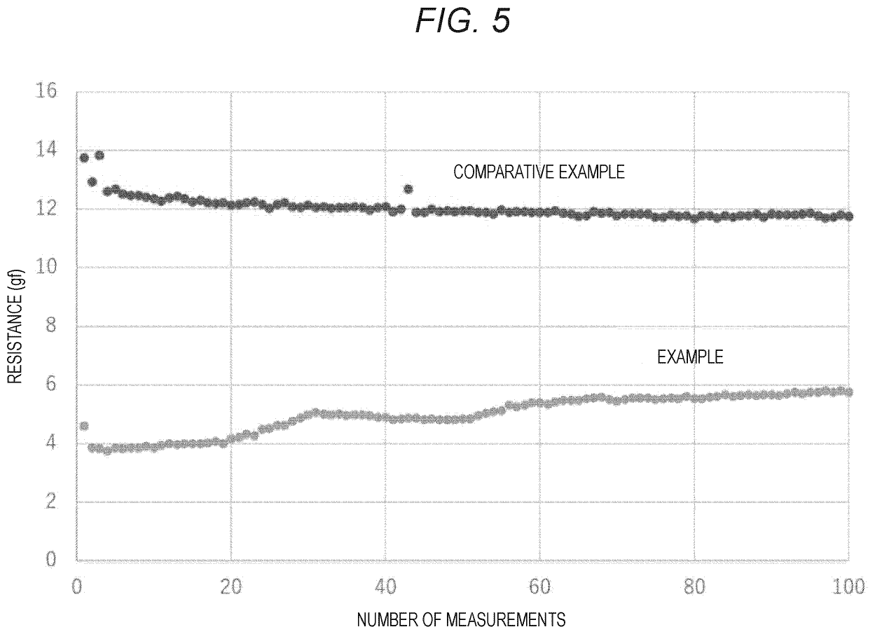

Hereinafter, with reference to FIG. 5, the elongated medical body disclosed here will be described in more detail through Example. Note that the technical scope of the invention is not limited to the following Example. FIG. 5 is a view illustrating resistances of medical elongated bodies according to Example and Comparative Example. In FIG. 5, the abscissa represents the number of measurements, and the ordinate represents the resistance.

### EXAMPLE

### Synthesis Example 1: Synthesis of Block Copolymer (1)

The following reaction was allowed to proceed, and a block copolymer (1) was manufactured.

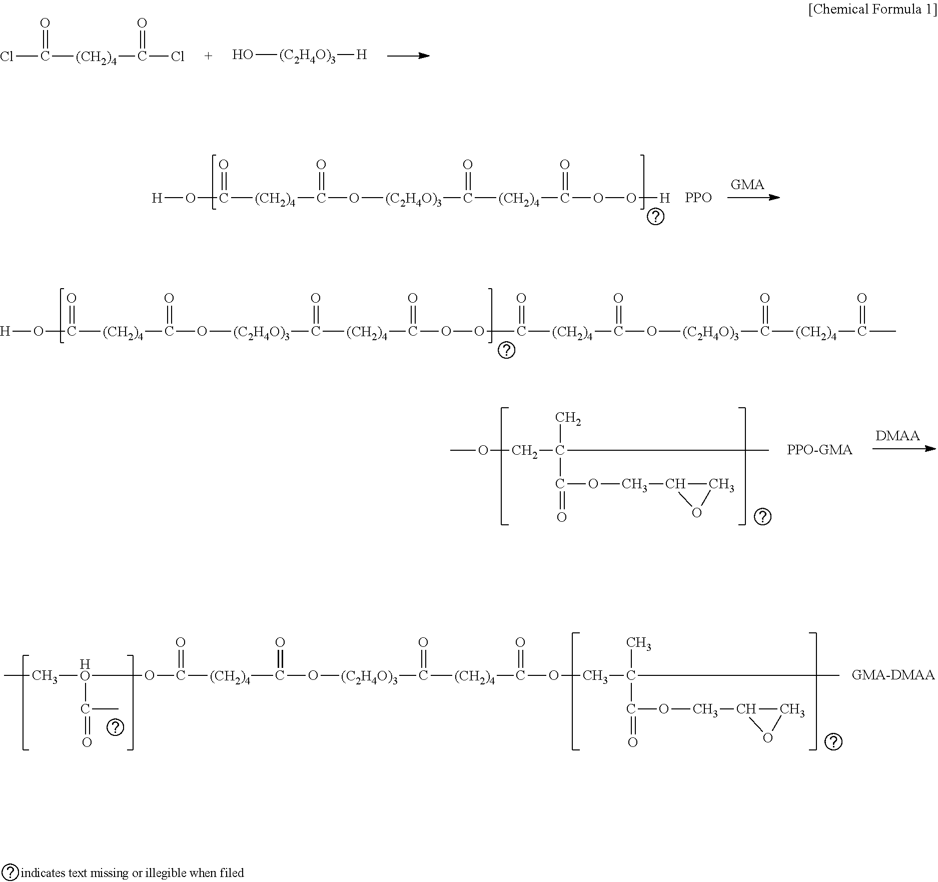

After 29.7 g of triethylene glycol was added dropwise to 72.3 g of adipic acid dichloride at 50° C., hydrochloric acid was removed under reduced pressure at 50° C. for 3 hours to obtain an oligoester.

Next, 4.5 g of methyl ethyl ketone was added to 22.5 g of the obtained oligoester, and the mixture was added dropwise to a solution containing 5 g of sodium hydroxide, 6.93 g of 31% hydrogen peroxide, 0.44 g of dioctyl phosphate as a surfactant, and 120 g of water and reacted at −5° C. for 20 minutes. The resulting product was repeatedly washed with water and methanol, and then, dried to obtain a poly peroxide (PPO) having a plurality of peroxide groups in a molecule.

Next, 0.5 g of this PPO, 9.5 g of glycidyl methacrylate (GMA), and also 30 g of benzene as a solvent were polymerized while being stirred at 80° C. for 2 hours under reduced pressure. The reactant obtained after the polymerization was reprecipitated with diethyl ether to obtain polyglycidyl methacrylate having a plurality of peroxide groups in a molecule (PPO-GMA).

Subsequently, 1.0 g of the obtained PPO-GMA (corresponding to 7 mmol of GMA) was charged into 9.0 g of N,N-dimethylacrylamide (DMAA) and 90 g of dimethyl sulfoxide as a solvent, and the mixture was reacted at 80° C. for 18 hours.

The reactant obtained after the reaction was reprecipitated with hexane and recovered, thereby obtaining a block copolymer (1) (constitutional unit (A):constitutional unit (B)=GMA:DMAA=1:14 (molar ratio)). The weight average molecular weight (Mw) of the block copolymer (1) measured by gel permeation chromatography (GPC, polystyrene equivalent) was about 1.5 million.

### Example 1: Production of Coated Tube (1)

A coating liquid was prepared by dissolving the block copolymer (1) obtained in Synthesis Example 1 and a polyvinyl chloride resin (available from FUJIFILM Wako Pure Chemical Corporation) in N, N-dimethylformamide (DMF) in such a manner that the mass ratio (first non-lubricating resin/first lubricating resin)=1/45 and the first non-lubricating resin in the coating liquid became 0.1 mass %. A tube having an outside diameter of 2.37 mm molded from a polyamide elastomer (second non-lubricating resin, hardness scale 60 Shore D, Vestamid E62, available from EVONIK) was immersed in the coating liquid and dried at room temperature (25° C.) for one hour to form a coating film. Furthermore, the coating film on the tube was heated in an oven at 110° C. for one hour, and then, cooled to room temperature. The elongated medical body 1 provided with the lubricant layer 14 was produced in this manner.

### Comparative Example 1

An elongated medical body was produced in a similar manner to Example 1 except that a lubricant layer was formed by heating at 130° C. for 12 hours and using a coating liquid with no poly vinyl chloride resin.

### Comparative Example 2

An elongated medical body was produced in a similar manner to Example 1 except that a lubricant layer was formed by heating at 110° C. for 1 hour and using a coating liquid with no poly vinyl chloride resin.

### [Results]

As illustrated in FIG. 5, the resistances of the elongated medical body according to Comparative Example 1 were above 10 gf, while the resistances of the elongated medical body 1 according to Example 1 were below 6 gf. The resistances measured in Comparative Example 2 were 8 gf but a resistance of the 23rd measurement increased sharply, and the test was aborted (not shown).

As described above, when the tube 10 is moved 100 times while clamped at 500 gf between the pair of abutting members 91 and 92, the resistance is constantly 10 gf or less. Accordingly, it is possible to provide an elongated medical body which has favorable pushability and trackability and maintains surface lubricity, that is, a small resistance due to friction, or does not decrease in surface lubricity even when subjected to friction and also reduces the number of fine particles generated.

The elongated medical body 1 according to the present invention has been described through the embodiment, but the invention is not limited to the configurations described in the embodiment and is appropriately changed based on the claims.

For example, in the embodiment, the elongated medical body 1 is used as a lower extremity guiding catheter, but the elongated medical body 1 may also be used as a catheter for angiographic catheter, microcatheter, guidewire support catheter, balloon catheter, stent delivery catheter, diagnostic imaging catheter, atherectomy catheter, introducer sheath, or dilator or may be used as a guidewire.

For example, in the embodiment, the outer layer 12 includes the second non-lubricating resin but may be a metallic core coated with a lubricant layer containing a lubricating resin to which a first non-lubricating resin is added. The metallic core may include a metallic member such as SUS or Ni—Ti. It is possible to employ various metallic materials, for example, stainless steels such as Ni—Ti-based alloys, SUS302, SUS304, SUS303, SUS316, SUS316L, SUS316J1, SUS316J1L, SUS405, SUS430, SUS434, SUS444, SUS429, and SUS430F, piano wires, cobalt alloys, and superelastic alloys.

The detailed description above describes embodiments of an elongated medical body and manufacturing method representing examples of the new elongated medical body and manufacturing method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An elongated medical body possessing a radially inwardly facing inner peripheral surface and a radially outwardly facing outer peripheral surface, the elongated medical body comprising:
an elongated tubular body possessing a proximal end and a distal end, the elongated tubular body having a lumen extending throughout a length of the elongated tubular body and opening to both an open distal end of the elongated tubular body at the distal end of the elongated tubular body and an open proximal end of the elongated tubular body at the proximal end of the elongated tubular body;
the elongated tubular body including a first layer extending along the length of the elongated tubular body;
the elongated tubular body including a second layer located at least at a distal end portion of the elongated tubular body, inclusive of the distal end of the elongated tubular body, the second layer being positioned radially outwardly of the first layer so that the second layer is positioned in overlying relation to and covers the first layer;
the second layer having an outer surface forming at least a part of the outer peripheral surface of the elongated medical body; and
the second layer being a lubricant layer comprised of a non-lubricating resin and a lubricating resin.

2. The elongated medical body according to claim 1, wherein the first layer is comprised of a non-lubricating resin different from the non-lubricating resin comprising the second layer.

3. The elongated medical body according to claim 1, wherein the lubricating resin in the second layer is a hydrophilic lubricating resin.

4. The elongated medical body according to claim 1, wherein the lubricating resin of the second layer is an acrylic resin.

5. The elongated medical body according to claim 1, further comprising a plurality of reinforcing wires embedded in the first layer.

6. The elongated medical body according to claim 1, further comprising an inner layer of fluororesin material positioned radially inwardly of the first layer so that the first layer is positioned in overlying relation to and covers the inner layer, the inner layer possessing an inner surface that is the inner peripheral surface of the elongated medical body.

7. An elongated medical body having a resistance that is constantly 10 gf or less when the elongated medical body is moved 100 times while the elongated medical body is clamped at 500 gf between a pair of abutting members.

8. The elongated medical body according to claim 7, wherein the elongated medical body is a tube.

9. The elongated medical body according to claim 7,
wherein the tube comprises:
a lubricant layer containing a lubricating resin to which a first non-lubricating resin is added; and
an outer layer coated with the lubricant layer and containing a second non-lubricating resin, and
a ratio of the first non-lubricating resin added to the lubricating resin ranges from 1/1000 to 1.

10. The elongated medical body according to claim 7, wherein the elongated medical body is guiding catheter for accessing a lower limb.

11. The elongated medical body according to claim 9, wherein the first non-lubricating resin is insoluble in water.

12. The elongated medical body according to claim 9, wherein the first non-lubricating resin is a vinyl chloride resin or a urethane resin.

13. The elongated medical body according to claim 9, wherein the second non-lubricating resin is a polyamide elastomer or a polyamide.

14. The elongated medical body according to claim 7,
wherein the tube comprises:
a lubricant layer containing a lubricating resin to which a first non-lubricating resin is added; and
a metallic member inside the lubricant layer, and
a ratio of the first non-lubricating resin added to the lubricating resin ranges from 1/1000 to 1.

15. The elongated medical body according to claim 14 wherein the metallic member is SUS or Ni—Ti.

16. The elongated medical body according to claim 15, wherein the elongated medical body is a guidewire.

17. The elongated medical body according to claim 9, wherein the lubricating resin is hydrophilic.

18. The elongated medical body according to claim 9, wherein the lubricating resin is an acrylic resin.

19. A method for manufacturing an elongated medical body, the method comprising:
coating an outer layer with a solution containing a first non-lubricating resin and a lubricating resin in which a mass ratio of the first non-lubricating resin added to the lubricating resin ranges from 1/1000 to 1 to produce a coated device, the outer layer coated with the solution containing a second non-lubricating resin; and
heating the coated device at 110° C. or less for 6 hours or less to form a lubricant layer on an outer periphery of the outer layer.

20. The method for manufacturing an elongated medical body according to claim 19, wherein the lubricant layer is formed on the outer periphery of the outer layer by heating the coated device for 1 hour or less.
